(19) United States
(12) Patent Application Publication
(10) Pub. No.: US 2025/0082354 A1
(43) Pub. Date: Mar. 13, 2025

(54) ENDOSCOPIC TRISECTING CUTTING SNARE SYSTEM

(71) Applicant: Mark Pankonin, Asheville, NC (US)

(72) Inventor: Mark Pankonin, Asheville, NC (US)

(21) Appl. No.: 18/466,191

(22) Filed: Sep. 13, 2023

(51) Int. Cl. A61B 17/32; A61B 1/018

(52) U.S. Cl. CPC A61B 17/320016; A61B 1/018; A61B 2017/0034; A61B 2017/00358

(57) ABSTRACT

An endoscopic trisecting cutting snare for use in colonoscopy or other endoscopic procedures, includes a primary snare loop, two trisecting cutting wires, a retracting wire, and a catheter for use in polypectomy or similar endoscopic procedures. The endoscopic trisecting cutting snare may be utilized in connection with an endoscopic device through a working channel for access to the operation site.

### FIELD OF THE INVENTION

This invention relates to an endoscopic snare and, more particularly, a system including a snare with a plurality of cross-filaments to create a trisecting cutting plane for the cutting and removal of tissue during a polypectomy.

### BACKGROUND OF THE INVENTION

Colonoscopy is the most commonly-performed gastroenterological procedures and is one of the most effective tools for colon cancer screening. A colonoscopy permits the direct visualization and removal of colon polyps, which are the precursors to colon cancers.

Colonoscopies are performed using a colonoscope: a long flexible tube featuring a camera and light source at the distal end of the device, along with two additional channel passages, or lumens. One of these channels is generally reserved for suctioning liquid and debris from the colon and into a collection container attached to the proximal end of a colonoscope. Tissue that is removed from the colon lining can thus be suctioned into the collection container for further analysis.

The second colonoscope channel is a working channel that permits passage of various therapeutic tools that can be used within the colon lumen, such as biopsy forceps, hemostatic clips, or plastic catheters containing either a cutting snare or a basket device for tissue retrieval.

A colonoscopy procedure involved insertion of the colonoscope in a patient's anus and advancement of the scope to the proximal-most part of the colon. The colonoscope is then slowly withdrawn while the gastroenterologist utilizes the camera and light source to examine the colon lining for polyps. If a polyp is detected, the gastroenterologist may perform a polypectomy utilizing either biopsy forceps or a snare.

Biopsy forceps are exclusively utilized for very small polyps measuring 2-3 mm in size. An endoscopic snare is generally required for polyps larger than 2-3 mm. Snare polypectomy utilizes a plastic catheter containing a thin metal snare loop, wherein the plastic catheter is inserted into the proximal colon via a channel in the colonoscope. Once the colonoscope and plastic catheter are optimally positioned, the snare loop is advanced out of the catheter and positioned around the polyp. A hand-held device is then utilized to retract the snare into the catheter, cutting the polyp from the colon's mucosal lining. After polypectomy, the subject tissue must be retrieved and sent to a pathologist for a histological diagnosis.

If the subject polyp is sufficiently small, it may simply be suctioned into the collection channel of the colonoscope and retrieved from the attached specimen container. Polyps too large to be suctioned into the collection channel (e.g., approximately 8 mm or greater) must be collected using one of two alternate methods. According to the first method, a catheter containing a basket device is utilized to collect the polyp. Once the polyp is enclosed within the basket, the entire colonoscope must be removed from the patient's colon while the catheter remains in place. Once the polyp is collected from the basket and the catheter is removed, the colonoscope must then be reinserted and advanced to the polypectomy site to continue the exam. This method is inefficient and adds significant time to the procedure, particularly where multiple large polyps must be removed. Similarly, this method increases the risk of complications due to the need for additional insertion and advancement of the colonoscope in the patient's colon, and it is of particular concern for cases in which there is atypical anatomy, prior proximal surgeries, and/or morbid obesity.

According to the second existing method, the same snare device utilized for the polypectomy is utilized in an attempt to cut the polyp into sufficiently small pieces to be suctioned into the collection channel and deposited into the specimen container. The snare device was not designed for such a procedure, and accordingly, it can be technically difficult and ineffective to attempt to grasp a free-floating polyp with the snare. This inefficient process can significantly prolong the procedure and/or can inhibit the ability to properly retrieve the polyp tissue for subsequent analysis. These complications may be exacerbated where visualization is poor due to poor colon preparation and retained stool.

These endoscopic techniques are similarly ineffective and inefficient for polypectomy and tissue retrieval from the stomach or small intestine by use of a gastroscope during an esophagogastroduodenoscopy (EGD). Gastric polyps are less common in practice, but they are often too large to be removed via a suction channel. Polypectomies in the small bowel further include unique challenges related to the limitations of the gastroscope length. For example, when a polyp is removed proximal to the maximum extension of the gastroscope, there is a risk of peristaltic contractions propagating the free-floating tissue distally to a location out of the gastroscope's reach, leading to inability to retrieve tissue during the time required to remove a snare and insert a collection basket.

Similarly, current systems and techniques for removal of esophageal obstructions of food are particularly ineffective and inefficient. For example, if the food obstruction cannot safely be pushed through into the stomach it must be removed via the mouth, and current means of collecting such food suffer from similar inefficiencies. Foreign bodies in the stomach may also be removed via endoscopic extraction, which is a preferable alternative to surgical removal. Grasping such foreign objects may be particularly difficult with a traditional, single snare that operates on a single plane.

These current techniques for polypectomy and tissue retrieval are sub-optimal for large colon polyps located in the proximal colon.

Therefore, there exists a need for an endoscopic tool that is capable of rapid, efficient, and safe polypectomy procedures and the subsequent retrieval of large polyps and polyp tissue from the proximal colon.

### SUMMARY OF THE INVENTION

In accordance with one form of the present invention, there is provided an endoscopic trisecting cutting snare system, the endoscopic trisecting cutting snare system including a filament forming a primary snare loop; two cutting wires, each cutting wire having two terminal ends, wherein each terminal end is attached to the primary snare loop; a retracting wire attached to the primary snare loop; a catheter having a distal end and a proximal end, wherein the catheter is sized for internal receipt of the retracting wire, the primary snare loop, and the at least two cutting wires; the endoscopic trisecting cutting snare system being selectively operable between a deployed configuration and a stored configuration; the deployed configuration defined by the primary snare loop and the at least two cutting wires being fully extended from within the catheter; and the stored configuration defined by the primary snare loop and the at least two cutting wires being at least partially retracted within the catheter.

In accordance with one form of the present invention, there is provided an endoscopic trisecting cutting snare system, including a filament forming a primary snare loop; two cutting wires, each cutting wire having two terminal ends, wherein each terminal end is attached to the primary snare loop; a retracting wire having a distal end and a proximal end, wherein the retracting wire is attached at its distal end to the primary snare loop; a catheter having a distal end and a proximal end, wherein the catheter is sized for internal receipt of the retracting wire, the primary snare loop, and the at least two cutting wires; a snare handle attached to the proximal end of the retracting wire, wherein the snare handle is configured to selectively operate the endoscopic trisecting cutting snare system between a deployed configuration and a stored configuration; the deployed configuration defined by the primary snare loop and the at least two cutting wires being fully extended from within the catheter; and the stored configuration defined by the primary snare loop and the at least two cutting wires being at least partially retracted within the catheter.

In accordance with one form of the present invention, there is provided a method of performing an endoscopic procedure, including providing an endoscope device having a distal end, a proximal end, at least one working channel, wherein the at least one working channel extends from the distal end to the proximal end; providing an endoscopic trisecting cutting snare system, including; a filament forming a primary snare loop; two cutting wires, each cutting wire having two terminal ends, wherein each terminal end is attached to the primary snare loop; a retracting wire attached to the primary snare loop; a catheter having a distal end and a proximal end, wherein the catheter is sized for internal receipt of the retracting wire, the primary snare loop, and the at least two cutting wires; the catheter being configured for receipt into the at least one working channel of the endoscope device; the endoscopic trisecting cutting snare system being selectively operable between a deployed configuration and a stored configuration; the deployed configuration defined by the primary snare loop and the at least two cutting wires being fully extended from within the catheter; the stored configuration defined by the primary snare loop and the at least two cutting wires being at least partially retracted within the catheter; inserting an endoscopic trisecting cutting snare system into the proximal end of the working channel of the endoscope device, whereby at least a portion of the endoscopic trisecting cutting snare system extends from the working channel at the distal end of endoscopic device; transitioning the endoscopic trisecting cutting snare system from the stored configuration to the deployed configuration; positioning the primary snare loop around a tissue object; and transitioning the endoscopic trisecting cutting snare system from the deployed configuration to the stored configuration.

### BRIEF DESCRIPTION OF THE DRAWINGS

For a fuller understanding of the nature of the present invention, reference should be made to the following detailed description, taken in conjunction with the accompanying drawings in which:

FIG. 1 is an isolated top view of a primary snare loop of an endoscopic trisecting cutting snare system in accordance with one embodiment;

FIG. 2 is an isolated side view of a primary snare loop of an endoscopic trisecting cutting snare system in accordance with one embodiment;

FIG. 3 is an isolated side perspective view of a primary snare loop of an endoscopic trisecting cutting snare system in accordance with one embodiment;

FIG. 4 is a top view of an endoscopic trisecting cutting snare system in accordance with one embodiment;

FIG. 5 is an isolated top view of a primary snare loop of an endoscopic trisecting cutting snare system in accordance with one embodiment;

FIG. 6 is an isolated side view of a primary snare loop of an endoscopic trisecting cutting snare system in accordance with one embodiment;

FIG. 7 is an isolated view of an endoscopic device, in accordance with one embodiment; and

FIG. 8 is an isolated view of an endoscopic device, and specifically, the working channel of an endoscopic device, in accordance with one embodiment;

FIG. 9 is an isolated top view of an endoscopic trisecting cutting snare system, in accordance with one embodiment; and

FIG. 10 is an isolated top view of an endoscopic trisecting cutting snare system, in accordance with one embodiment.

Like reference numerals refer to like parts throughout the several views of the drawings.

### DETAILED DESCRIPTION OF THE PREFERRED EMBODIMENTS

Referring to the several views of the drawings, the endoscopic trisecting cutting snare system 100 of the present invention is shown and described herein and is generally indicated as 100.

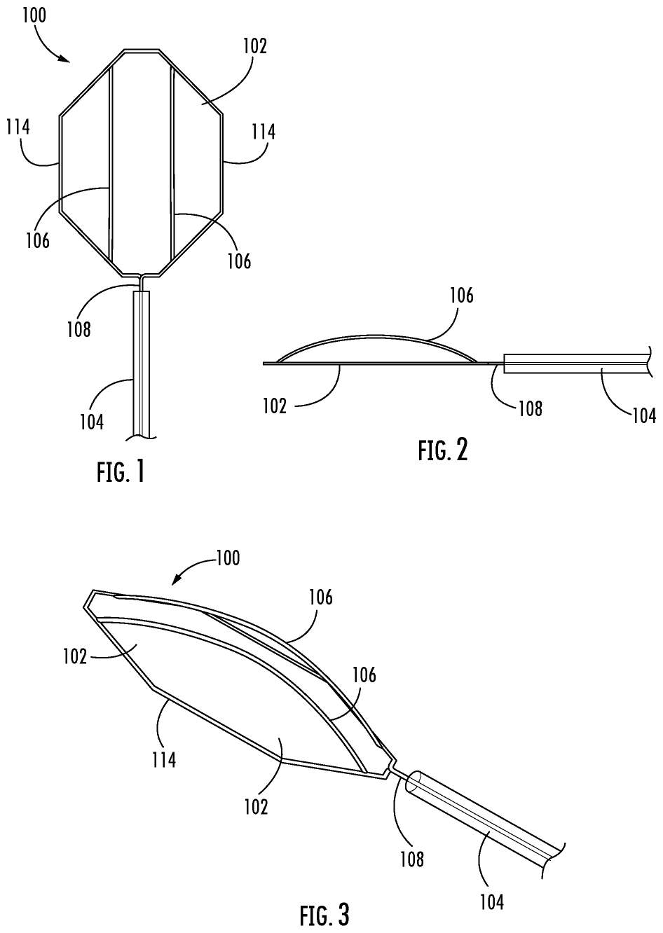

Referring initially to FIGS. 1-2, the endoscopic trisecting cutting snare system 100 includes a primary snare loop 102, at least two cutting wires 106, a catheter 104, wherein the primary snare loop 102 and at least two cutting wires 106 are designed to be removably housed within the catheter 104. The primary snare loop 102 may be attached to a retracting wire 108, wherein the retracting wire 108 may extend through the length of the catheter 104 and the retracting wire's 108 opposing end may be engaged with a snare handle 110, thus connecting the primary snare loop 102 to the snare handle 110 via the retracting wire 108. The snare handle 110 may be configured to operate in deploying the primary snare loop 102 and cutting wires 106 from the catheter 104. The catheter 104, in turn, is sized and configured to pass through a working channel of an endoscopic device 114 to permit the primary snare loop 102 and cutting wires 106 to be deployed within, for example, a patient's colon. It will be appreciated by one of ordinary skill in the art that the endoscopic trisecting cutting snare system 100 may be used in additional similar environments, including in esophageal and/or stomach applications,

According to one embodiment, the cutting wires 106 may be soldered onto the primary snare loop 102 or the cutting wires 106 may be integrally formed with the primary snare loop 102. According to one embodiment, the primary snare loop 102 and/or the cutting wires 106 may be composed of platinum, a platinum alloy, stainless steel, a stainless steel alloy, and/or other similar material(s). According to one embodiment, the catheter 104 may be composed of plastic, a plastic polymer, or similar material that is sufficiently resilient, durable, and flexible for purposes of use of the invention.

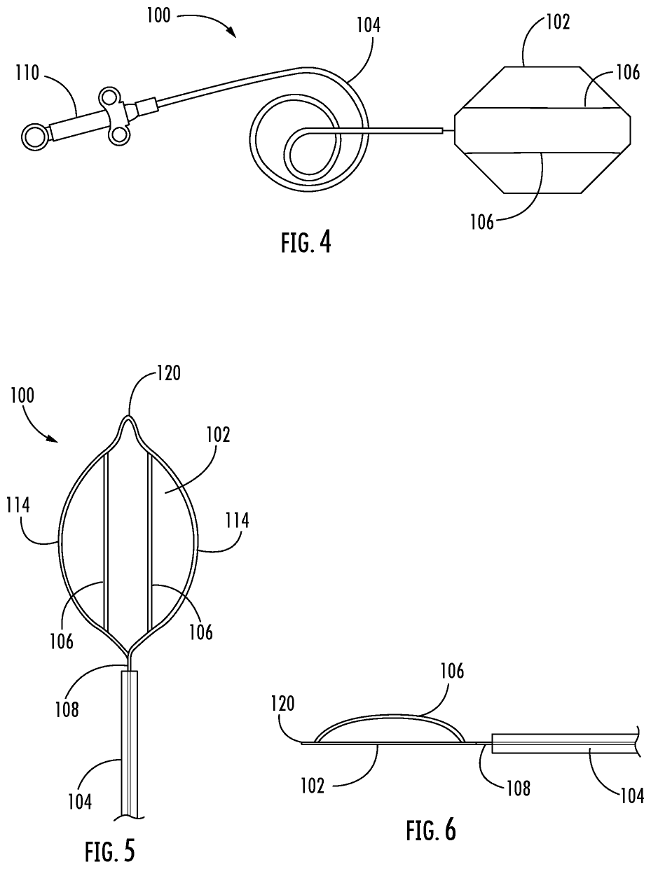

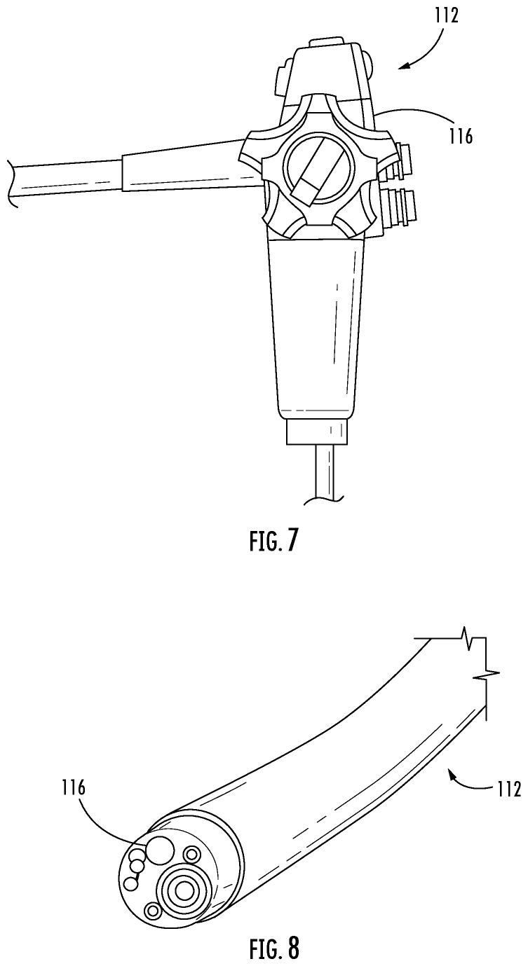

According to one embodiment, in a stored state, the primary snare loop 102 and the cutting wires 106 are stored inside the catheter 104 until the endoscopic trisecting cutting snare system 100 is transitioned into a deployed state. For example, and as shown in FIG. 4, a snare handle 110 may be engaged to deploy the primary snare loop 102 and cutting wires 106 from the catheter 104. Engagement of the snare handle 110 may impart a longitudinal force along the retracting wire 108 to push the primary snare loop 102 and cutting wires 106 out of the end of the catheter 104. Similarly, the snare handle 110 may be engaged in an alternate manner to impart a tensional force along the retracting wire 108 to retrieve the primary snare loop 102 and cutting wires 106 into the catheter 104.

According to one embodiment, and as shown in FIGS. 1, 5, and 9-10, the cutting wires 106 may be positioned equidistant from one another and/or from the snare lateral edges 114.

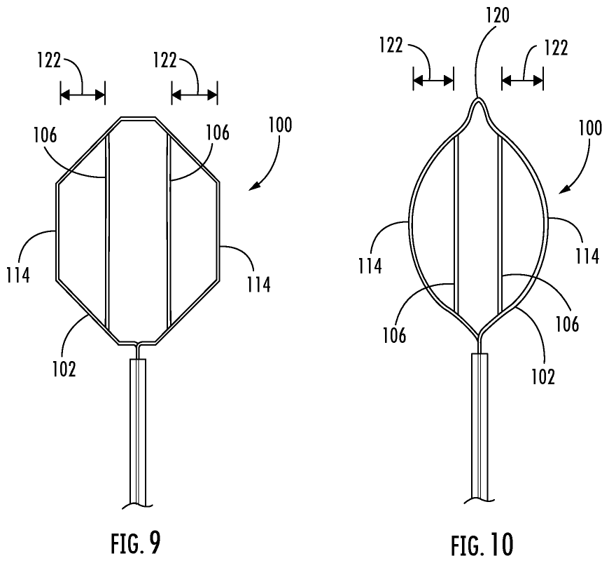

According to one embodiment, and as shown in FIGS. 9-10, the endoscopic trisecting cutting snare system 100 may have two lateral distances 122. The lateral distances 122 are equal to the distance between a cutting wire's 106 midpoint (measured as the distance approximately halfway between a cutting wire's attachment points to the primary snare loop) and a parallel tangent located on the closest snare lateral edge 114 of the primary snare loop 102. According to one embodiment, the two lateral distances 122 are equal or are substantially equal.

For example, the primary snare loop 102 may have a total width of approximately 2.4 cm-2.5 cm, measured between the snare lateral edges 114. The primary snare loop 102 may have a total length of approximately 3.0 cm. Each cutting wire 106 may be positioned approximately 0.8 cm from the nearest snare lateral edge (forming a lateral distance 122) and approximately 0.8 cm-0.9 cm from the other cutting wire 106.

In operation, once the endoscopic device 112 is properly positioned within a patient's colon, a user inserts a catheter 104 housing the primary snare loop 102, cutting wires 106, and retracting wire 108 into the patient's colon via the working channel 116 of the endoscopic device 112. The user may then manipulate the snare handle 110 to transition the endoscopic trisecting cutting snare system 100 into the deployed configuration, thus extending the primary snare loop 102 and cutting wires 106 from the catheter 104.

Once the endoscopic trisecting cutting snare system 100 is in the deployed configuration, the primary snare loop 102 and cutting wires 106 may be positioned over the polyp (or other tissue material) to be removed. The arrangement of the cutting wires 106 and primary snare loop 102 permits unimpeded visualization of the polyp and surrounding tissue, allowing a user to ensure complete manipulation and/or removal of the polyp.

After securing the primary snare loop 102 around the site at which a polyp will be cut, a user may retract the primary snare loop 102 and cutting wires 106 into the catheter 104 by, for example, manipulation of a snare handle 110. By retracting the primary snare loop 102 and cutting wires 106 into the catheter 104, the polyp is simultaneously removed from the colon lining and cut into three smaller fragments that can be immediately suctioned into a suction channel and from a suction channel deposited into a collection chamber. For exceptionally large polyps, the primary snare loop 102 and cutting wires 106 may be repositioned to perform additional cutting of the polyp for collection via the suction channel.

It will be appreciated by one of ordinary skill in the art that the endoscopic trisecting cutting snare system 100 may be adapted to fit a variety of endoscopic device 112 designs without departing from the spirit or scope of the invention.

According to one embodiment, and as shown in FIGS. 5-6, the primary snare loop 102 may be a closed curved shape and may include a snare tip 120 located, for example, substantially opposite the point of intersection between the primary snare loop 102 and the retracting wire 108. The snare tip 120 is configured as a small channel formed by angled bends in the primary snare loop. The snare tip 120 may function to assist in anchoring the primary snare loop 102 around a polyp (or other tissue to be removed) for precision removal of the polyp.

According to one embodiment, and as shown in FIGS. 5-6, in the deployed state, the primary snare loop 102 may be a closed curved shape with no straight-line edges.

It will be appreciated by one of ordinary skill in the art that the endoscopic trisecting cutting snare system 100 may be adapted for use in polypectomy and tissue retrieval from the stomach or small intestine with a gastroscope during an esophagogastroduodenoscopy (EGD). Use of the endoscopic trisecting cutting snare system's 100 primary snare loop 102 and cutting wires 106 obviates the need for a basket device for retrieval or for the inefficient and ineffective use of a traditional single-plane snare for attempted cutting and retrieval.

For example, use of the endoscopic trisecting cutting snare system 100 enables polypectomies in the stomach and/or small intestine, with immediate collection of the tissue and minimization of the risk of tissue loss.

Similarly, it will be appreciated by one of ordinary skill in the art that the endoscopic trisecting cutting snare system 100 may be used for removal of foreign bodies in the esophagus, stomach, small bowel, and colon, particularly where such objects may be of an irregular size and/or shape. For example, the primary snare loop 102 and the cutting wires 106 may serve as a collection basket where they serve as three points of contact for ensnaring the foreign object. Once the foreign object is ensnared, the user may transition the endoscopic trisecting cutting snare system 100 from the deployed state to the stored state to grasp and retain the foreign object for retrieval.

While the present invention has been shown and described in accordance with several preferred and practical embodiments, it is recognized that departures from the instant disclosure are contemplated within the spirit and scope of the present invention.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For brevity and/or clarity, well-known functions or constructions may not be described in detail herein.

The term “exemplary” is used herein to mean serving as an example, instance, or illustration. Any aspect or design described herein as “exemplary” is not necessarily to be construed as preferred or advantageous over other aspects or designs. Similarly, examples are provided herein solely for purposes of clarity and understanding and are not meant to limit the subject innovation or portion thereof in any manner.

The terms “for example” and “such as” mean “by way of example and not of limitation.” The subject matter described herein is provided by way of illustration for the purposes of teaching, suggesting, and describing, and not limiting or restricting. Combinations and alternatives to the illustrated embodiments are contemplated, described herein, and set forth in the claims.

For convenience of discussion herein, when there is more than one of a component, that component may be referred to herein either collectively or singularly by the singular reference numeral unless expressly stated otherwise or the context clearly indicates otherwise. For example, components N (plural) or component N (singular) may be used unless a specific component is intended. Also, the singular forms “a,” “an,” and “the” are intended to include the plural forms as well, unless expressly stated otherwise or the context indicates otherwise.

It will be further understood that the terms “includes,” “comprises,” “including,” and/or “comprising” specify the presence of stated features, steps, operations, elements, and/or components, but do not preclude the presence or addition of one or more other features, steps, operations, elements, components, and/or groups thereof unless explicitly stated otherwise or the context clearly requires otherwise. The terms “includes,” “has” or “having” or variations in form thereof are intended to be inclusive in a manner similar to the term “comprises” as that term is interpreted when employed as a transitional word in a claim.

It will be understood that when a component is referred to as being “connected” or “coupled” to another component, it can be directly connected or coupled or coupled by one or more intervening components unless expressly stated otherwise or the context clearly indicates otherwise.

The term “and/or” includes any and all combinations of one or more of the associated listed items. As used herein, phrases such as “between X and Y” and “between about X and Y” should be interpreted to include X and Y unless expressly stated otherwise or the context clearly indicates otherwise.

Terms such as “about”, “approximately”, and “substantially” are relative terms and indicate that, although two values may not be identical, their difference is such that the apparatus or method still provides the indicated or desired result, or that the operation of a device or method is not adversely affected to the point where it cannot perform its intended purpose. As an example, and not as a limitation, if a height of “approximately X inches” is recited, a lower or higher height is still “approximately X inches” if the desired function can still be performed or the desired result can still be achieved.

While the terms vertical, horizontal, upper, lower, bottom, top, and the like may be used herein, it is to be understood that these terms are used for ease in referencing the drawing and, unless otherwise indicated or required by context, does not denote a required orientation.

The different advantages and benefits disclosed and/or provided by the implementation(s) disclosed herein may be used individually or in combination with one, some or possibly even all of the other benefits. Furthermore, not every implementation, nor every component of an implementation, is necessarily required to obtain, or necessarily required to provide, one or more of the advantages and benefits of the implementation.

Conditional language, such as, among others, “can”, “could”, “might”, or “may”, unless specifically stated otherwise, or otherwise understood within the context as used, is generally intended to convey that certain embodiments preferably or optionally include certain features, elements and/or steps, while some other embodiments optionally do not include those certain features, elements and/or steps. Thus, such conditional language indicates, in general, that those features, elements and/or step may not be required for every implementation or embodiment.

The subject matter described herein is provided by way of illustration only and should not be construed as limiting the nature and scope of the subject invention. While examples of aspects of the subject invention have been provided above, it is not possible to describe every conceivable combination of components or methodologies for implementing the subject invention, and one of ordinary skill in the art may recognize that further combinations and permutations of the subject invention are possible. Furthermore, the subject invention is not necessarily limited to implementations that solve any or all disadvantages which may have been noted in any part of this disclosure. Various modifications and changes may be made to the subject invention described herein without following, or departing from the spirit and scope of, the exemplary embodiments and applications illustrated and described herein. Although the subject matter presented herein has been described in language specific to components used therein, it is to be understood that the subject invention is not necessarily limited to the specific components or characteristics thereof described herein; rather, the specific components and characteristics thereof are disclosed as example forms of implementing the subject invention. Accordingly, the disclosed subject matter is intended to embrace all alterations, modifications, and variations, that fall within the scope and spirit of any claims that are written, or may be written, for the subject invention.

What is claimed is:

1. An endoscopic trisecting cutting snare system comprising:
a filament forming a primary snare loop;
two cutting wires, each cutting wire having two terminal ends, wherein each terminal end is attached to the primary snare loop;
a retracting wire attached to the primary snare loop;
a catheter having a distal end and a proximal end, wherein the catheter is sized for internal receipt of the retracting wire, the primary snare loop, and the at least two cutting wires;
the endoscopic trisecting cutting snare system being selectively operable between a deployed configuration and a stored configuration;
the deployed configuration defined by the primary snare loop and the at least two cutting wires being fully extended from within the catheter; and
the stored configuration defined by the primary snare loop and the at least two cutting wires being at least partially retracted within the catheter.

2. The endoscopic trisecting cutting system of claim 1, wherein:
in the deployed configuration, the primary snare loop forms an octagonal shape.

3. The endoscopic trisecting cutting snare system of claim 1, wherein the primary snare loop includes a snare tip.

4. The endoscopic trisecting cutting snare system of claim 3, wherein the snare tip is located in a position on the primary snare loop that is approximately one hundred eighty degrees (180°) relative to the point of intersection between the primary snare loop and the retracting wire.

5. The endoscopic trisecting cutting snare system of claim 1, wherein in the deployed configuration:
the primary snare loop forms a primary plane;
each cutting wire is substantially non-linear;
each cutting wire forms a primary cutting wire plane;
wherein each cutting wire plane is non-parallel to the primary plane.

6. The endoscopic trisecting cutting snare system of claim 1, wherein in the deployed configuration:
each cutting wire is substantially non-linear;
each cutting wire forms a primary cutting wire plane;
wherein the cutting wire planes are substantially parallel.

7. The endoscopic trisecting cutting snare system of claim 6, further comprising:
each cutting wire having a cutting wire midpoint defined by a point along the cutting wire that is equidistant to the terminal ends of the cutting wire;
the primary snare loop having two snare lateral edges, wherein each snare lateral edge is defined by a tangent parallel to each cutting wire plane;
a lateral distance defined by the distance between each cutting wire midpoint and the proximate snare lateral edge, measured perpendicular to the respective cutting wire;
wherein in the deployed configuration, each lateral distance is substantially equal.

8. An endoscopic trisecting cutting snare system comprising:
a filament forming a primary snare loop;
two cutting wires, each cutting wire having two terminal ends, wherein each terminal end is attached to the primary snare loop;
a retracting wire having a distal end and a proximal end, wherein the retracting wire is attached at its distal end to the primary snare loop;
a catheter having a distal end and a proximal end, wherein the catheter is sized for internal receipt of the retracting wire, the primary snare loop, and the at least two cutting wires;
a snare handle attached to the proximal end of the retracting wire, wherein the snare handle is configured to selectively operate the endoscopic trisecting cutting snare system between a deployed configuration and a stored configuration;
the deployed configuration defined by the primary snare loop and the at least two cutting wires being fully extended from within the catheter; and
the stored configuration defined by the primary snare loop and the at least two cutting wires being at least partially retracted within the catheter.

9. The endoscopic trisecting cutting snare system of claim 8, wherein:
in the deployed configuration, the primary snare loop forms an octagonal shape.

10. The endoscopic trisecting cutting snare system of claim 8, wherein the primary snare loop includes a snare tip.

11. The endoscopic trisecting cutting snare system of claim 9, wherein the snare tip is located in a position on the primary snare loop that is approximately one hundred eighty degrees (180°) relative to the point of intersection between the primary snare loop and the retracting wire.

12. The endoscopic trisecting cutting snare system of claim 8, wherein in the deployed configuration:
the primary snare loop forms a primary plane;
each cutting wire is substantially non-linear;
each cutting wire forms a primary cutting wire plane;
wherein each cutting wire plane is non-parallel to the primary plane.

13. The endoscopic trisecting cutting snare system of claim 8, wherein in the deployed configuration:
each cutting wire is substantially non-linear;
each cutting wire forms a primary cutting wire plane;
wherein the cutting wire planes are substantially parallel.

14. The endoscopic trisecting cutting snare system of claim 13 further comprising:
each cutting wire having a cutting wire midpoint defined by a point along the cutting wire that is equidistant to the terminal ends of the cutting wire;
the primary snare loop having two snare lateral edges, wherein each snare lateral edge is defined by a tangent parallel to each cutting wire plane;
a lateral distance defined by the distance between each cutting wire midpoint and the proximate snare lateral edge, measured perpendicular to the respective cutting wire;
wherein in the deployed configuration, each lateral distance is substantially equal.

15. A method of performing an endoscopic procedure, the method comprising:
providing an endoscope device having a distal end, a proximal end, at least one working channel, wherein the at least one working channel extends from the distal end to the proximal end;
providing an endoscopic trisecting cutting snare system, including;
a filament forming a primary snare loop;
two cutting wires, each cutting wire having two terminal ends, wherein each terminal end is attached to the primary snare loop;
a retracting wire attached to the primary snare loop;
a catheter having a distal end and a proximal end, wherein the catheter is sized for internal receipt of the retracting wire, the primary snare loop, and the at least two cutting wires;
the catheter being configured for receipt into the at least one working channel of the endoscope device;
the endoscopic trisecting cutting snare system being selectively operable between a deployed configuration and a stored configuration;
the deployed configuration defined by the primary snare loop and the at least two cutting wires being fully extended from within the catheter;
the stored configuration defined by the primary snare loop and the at least two cutting wires being at least partially retracted within the catheter;
inserting an endoscopic trisecting cutting snare system into the proximal end of the working channel of the endoscope device, whereby at least a portion of the endoscopic trisecting cutting snare system extends from the working channel at the distal end of endoscopic device;
transitioning the endoscopic trisecting cutting snare system from the stored configuration to the deployed configuration;
positioning the primary snare loop around a tissue object; and
transitioning the endoscopic trisecting cutting snare system from the deployed configuration to the stored configuration.

16. The method of claim 15, wherein in the deployed configuration, the primary snare loop forms an octagonal shape.

17. The method of claim 15, further comprising:
the primary snare loop including a snare tip, wherein the snare tip is located in a position on the primary snare loop that is approximately one hundred eighty degrees (180°) relative to the point of intersection between the primary snare loop and the retracting wire.

18. The method of claim 15, wherein in the deployed configuration:
the primary snare loop forms a primary plane;
each cutting wire is substantially non-linear;
each cutting wire forms a primary cutting wire plane;
each cutting wire plane being non-parallel to the primary plane.

19. The method of claim 15, wherein in the deployed configuration:
each cutting wire is substantially non-linear;
each cutting wire forms a primary cutting wire plane;
wherein the cutting wire planes are substantially parallel.

20. The method of claim 19, further comprising:
each cutting wire having a cutting wire midpoint defined by a point along the cutting wire that is equidistant to the terminal ends of the cutting wire;
the primary snare loop having two snare lateral edges, wherein each snare lateral edge is defined by a tangent parallel to each cutting wire plane;
the primary snare loop having two lateral distances, each lateral distance being defined by the distance between each cutting wire midpoint and the proximate snare lateral edge, measured perpendicular to the respective cutting wire;
wherein in the deployed configuration, each lateral distance is substantially equal.
